(19) United States
(12) Patent Application Publication
(10) Pub. No.: US 2005/0183213 A1
(43) Pub. Date: Aug. 25, 2005

(54) Composition for dyeing keratin fibers, comprising at least one alcohol oxidase and at least one quinone direct dye, and process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,322

(22) Filed: Jan. 28, 2005

Related U.S. Application Data
(60) Provisional application No. 60/545,923, filed on Feb. 20, 2004.

(30) Foreign Application Priority Data
Jan. 28, 2004 (FR) 0400785

(51) Int. Cl. A61K 7/13

(52) U.S. Cl. 8/406

(57) ABSTRACT

Disclosed herein is a composition for dyeing keratin fibers, for example human keratin fibers such as the hair, comprising, in a medium that is suitable for dyeing keratin fibers, at least one oxidation dye precursor, at least one alcohol oxidase enzyme, at least one substrate for the at least one alcohol oxidase enzyme and at least one quinone direct dye. Also disclosed is a process for dyeing keratin fibers, which comprises applying this composition to keratin fibers. Also disclosed is a multi-compartment device comprising the composition.

This application claims benefit of U.S. Provisional Application No. 60/545,923, filed Feb. 20, 2004.

Disclosed herein is a composition for dyeing keratin fibers, for example, human keratin fibers such as the hair, comprising, in a medium that is suitable for dyeing, at least one oxidation dye precursor, at least one alcohol oxidase enzyme, at least one substrate for the enzyme, and also one quinone direct dye.

It is known practice to dye keratin fibers, such as human hair, with compositions comprising oxidation dye precursors, for example, ortho- or para-phenylenediamines, ortho- or para-aminophenols, and heterocyclic compounds, which are generally referred to as oxidation bases. These oxidation bases, are colorless or weakly colored compounds which, when combined with oxidizing products, may give rise to colored compounds by a process of oxidative condensation.

It is also known that the shades obtained with these oxidation bases may be varied by combining them with couplers or coloration modifiers, the latter being chosen, for example, from aromatic meta-diamines, meta-aminophenols, meta-diphenols and certain heterocyclic compounds such as indole compounds.

The variety of molecules used as oxidation bases and couplers makes it possible to obtain a wide range of colors.

The “permanent” coloration obtained by these oxidation dyes should strive to achieve several goals. For example, it should have no toxicological drawbacks and it should also allow shades of the desired intensity to be obtained and have good resistance to external agents, including light, bad weather, washing, permanent waving, perspiration and rubbing.

The dyes should also ideally allow white hairs to be covered and, lastly, they should ideally be as unselective as possible, that is to say that they ideally should allow the smallest possible differences in coloration to be produced over the entire length of the same keratin fiber, which is generally differently sensitized (i.e. damaged) between its end and its root.

The dyeing is generally performed in strongly alkaline medium, in the presence of hydrogen peroxide. However, the use of alkaline media in the presence of hydrogen peroxide has the possible drawback of causing considerable degradation of the fibers, and also bleaching of keratin fibers, which is not always desirable.

Furthermore, this type of composition has the possible drawback of having to prepare a mixture between hydrogen peroxide and the dye support at the time of application of the composition to the keratin fibers.

The oxidation dyeing of keratin fibers may also be performed using oxidizing systems other than hydrogen peroxide, such as enzymatic systems, for example, with enzymes of the 2-electron oxidase type. Thus, French patent application FR 2 769 219 describes the use of a uricase enzyme and of its uric acid substrate in oxidation dyeing to dye keratin fibers. These enzymes catalyse the oxidation of a substrate via atmospheric oxygenation to generate one or more oxidation products, and also hydrogen peroxide. The hydrogen peroxide generated may be used to oxidize oxidation dye precursors and, consequently, to produce color on the hair. This system makes it possible to envisage oxidation dyeing without mixing at the time of use. However, although the dye formulations using alcohol oxidase are used under conditions that do not result in degradation of the hair comparable to that generated by formulations using hydrogen peroxide, and although offering the possibility of being formulated all-in-one, they lead to colorations that are may be insufficient as regards both the homogeneity of the color, the dyeing power and the chromaticity.

Patent application EP-A-0 310 675 describes the use of oxidation dye precursors of benzenic type in combination with enzymes such as pyranose oxidase. The compositions described in this patent may also comprise direct dyes. However, the colorations obtained using these compositions may be unsatisfactory.

Disclosed herein are novel compositions for dyeing keratin fibers by oxidation dyeing, which respect the nature of the keratin fiber, which offer the possibility of being formulated all-in-one and which may lead to homogeneous, strong colors and high chromaticity.

Disclosed herein are novel compositions comprising at least one oxidation dye precursor, at least one alcohol oxidase enzyme, at least one substrate for the at least one alcohol oxidase enzyme, and at least one quinone direct dye. These compositions can produce dyeing results with strong, chromatic unselective and fast colors. In addition, these compositions are capable of generating varied shades of intense and uniform color, without any significant degradation of the hair.

Other characteristics, aspects, subjects and advantages of the disclosed compositions will emerge even more clearly on reading the description and the examples that follow.

The at least one quinone direct dye that may be used are dyes of benzoquinone, naphthoquinone and anthraquinone type. The anthraquinone dyes may be neutral, acidic or cationic. Exemplary embodiments of the quinone direct dyes are the following dyes: Disperse Red 15, Solvent Violet 13, Acid Violet 43 (C.I. 60730), Disperse Violet 1, Disperse Violet 4, Disperse Blue 1, Disperse Violet 8, Disperse Blue 3, Disperse Red 11, Acid Blue 25 (C.I. 62055), Acid Blue 62 (C.I. 62045), Acid Blue 78 (C.I. 62105), Disperse Blue 7, Basic Blue 22, Disperse Violet 15, Basic Blue 99, Mordant Red 3 (C.I. 58005), Acid Green 25 (C.I. 61570), N-methylmorpholinium-propylamino-4-hydroxyanthraquinone, 1-aminopropylamino-4-methylaminoanthraquinone, 1-aminopropylaminoanthra-quinone, 5-β-hydroxyethyl-1,4-diaminoanthraquinone, 2-aminoethylaminoanthraquinone, 1,4-bis(β,γ-dihydroxy-propylamino)anthraquinone, 2,5-dihydroxybenzoquinone, 2-hydroxy-3-methoxynaphthoquinone, and 2,3-dihydroxy-naphthoquinone, and also quinone natural dyes, such as lawsone, juglone, alizarin, purpurin, spinulosin, 2,5-dihydroxynaphthoquinone, carminic acid and kermesic acid.

Most of these dyes are described in the Color Index published by The Society of Dyers and Colorists, P.O. Box 244, Perkin House, 82 Grattan Road, Bradford, Yorkshire, BD1 2JBN, England.

The at least one quinone direct dyes may be present in the composition at a concentration ranging from 0.001% to 20% by weight, relative to the total weight of the composition, or may be present in the composition at a concentration ranging from 0.005% to 10% by weight, relative to the total weight of the composition.

The at least one alcohol oxidase enzyme used in the composition may belong to the sub-subclass E.C.1.1.3 of the enzyme nomenclature (see Enzyme Nomenclature, Academic Press Inc; 1992).

The at least one alcohol oxidase enzyme may be chosen from primary alcohol oxidases (EC1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

In one embodiment, the enzyme used in the composition according to the invention, is a primary alcohol oxidase (EC 1.1.3.13).

Alcohol oxidase enzymes form a particular class of 2-electron oxidoreductase enzymes.

The at least one alcohol oxidase enzyme may be derived from an extract of plants, of animals, of micro-organisms (bacterium, fungus, yeast, microalga or virus), of differentiated or undifferentiated cells, obtained in vivo or in vitro, unmodified or genetically modified, or synthetic (obtained by chemical or biotechnological synthesis).

For example, the at least one alcohol oxidase enzyme may be extracted from the following species: Pinus, Gastropode, Manduca, Pichia, Candida, Pleurotus, Pseudomonas, Rhodococcus, Aspergillus, Kamagataella, Phanerochaete, Polyporus, Hansenula, Poria and Penicillium. In other exemplary embodiments, the at least one alcohol oxidase enzyme may be extracted from: Pinus strobus, which is a species of plant origin, Gastropode mollusc and Manduca sexta, which are of animal origin, Pichia sp. (pastoris, methanolica, angusta) and Candida sp. (boidinii, albicans, tropicalis), which are yeasts, Pleurotus pulmonarius, Aspergillus niger, Kamagataella pastoris, Phanerochaete chrysosporium, Polyporus obtusus, Hansenula polymorpha, Poria contigua, Penicillium simplicissimum, which are fungi, and Pseudomonas pseudoalcaligenes and Rhodococcus erythropolis, which are bacteria.

In an exemplary embodiment, the oxidase alcohol used comes from the Pichia pastoris strain.

Generally, the concentration of the at least one alcohol oxidase enzyme used in the composition ranges from 0.05% to 20% by weight, relative to the total weight of the composition. In another exemplary embodiment, the concentration ranges from 0.1% to 10%, such as from 0.5% to 8% by weight, relative to the total weight of the composition.

The enzymatic activity of the at least one alcohol oxidase enzymes may be defined from the oxidation of the donor under aerobic conditions. One unit U corresponds to the amount of enzyme leading to the generation of 1 μmol of hydrogen peroxide per minute at a given pH and at a temperature of 25° C.

The amount of the at least one alcohol oxidase enzyme used in the composition may, for example, range from 10³U to 10⁵U per 100 g of composition, such as from 2×10³U to 2×10⁴U per 100 g of composition.

The at least one substrate for the at least one alcohol oxidase enzyme can also be known as donors for the enzyme. The nature of the at least one substrate may vary as a function of the nature of the at least one alcohol oxidase enzyme which is used. For example, the substrate for the alcohol oxidase enzyme in the composition comprises an alcohol chosen from branched and unbranched, saturated and unsaturated, substituted and unsubstituted primary alcohols, secondary alcohols, long-hydrocarbon-chain alcohols, and aromatic alcohols. For example, donors for the primary alcohol oxidases that may be mentioned include primary alcohols containing from 1 to 6 carbon atoms. Donors for the aryl alcohol oxidases that may be mentioned include benzyl alcohol, 4-tert-butylbenzyl alcohol, 3-hydroxy-4-methoxybenzyl alcohol, veratryl alcohol, 4-methoxy-benzyl alcohol and cinnamyl alcohol. 2,4-hexadien-1-ol may also be used as donor for the aryl alcohol oxidases.

According to another exemplary embodiment, the at least one substrate for the at least one alcohol oxidase enzyme may be a compound bearing at least one aliphatic or aromatic alcohol function, which is suitable for reacting with the enzyme used. The compound bearing at least one aliphatic or aromatic alcohol function may, for example, be an oxidation dye precursor or alternatively a cosmetically acceptable adjuvant, for example a polymer, a surfactant or a preserving agent bearing at least one alcohol function. In another exemplary embodiment, the at least one substrate for the at least one alcohol oxidase enzyme is an oxidation dye precursor bearing at least one aliphatic or aromatic alcohol function. For example, N-(β-hydroxypropyl)-para-phenylenediamine, which bears a primary alcohol function, may serve as oxidation base and as substrate for the alcohol oxidase. Similarly, oxidation couplers, such as meta- or para-aminophenol, may fulfil both functions. Such precursors are described herein below. In this embodiment, the use of other substrates for the enzyme is optional.

Thus, one exemplary embodiment is a composition for dyeing keratin fibers, for example human keratin fibers such as the hair, comprising, in a medium that is suitable for dyeing: at least one oxidation dye precursor; at least one alcohol oxidase enzyme; at least one substrate, bearing an alcohol function, for the at least one alcohol oxidase enzyme, and at least one quinone direct dye. The at least one substrate may be totally or partially replaced with the at least one oxidation dye precursor if the at least one oxidation dye precursor comprises at least one aliphatic or aromatic alcohol functional group.

The use of the composition can reduce the risks associated with the handling of hydrogen peroxide. Furthermore, the concentration of preserving agents in the compositions according to the present disclosure may be reduced by supplying compounds containing an alcohol function which also have preserving properties.

In an exemplary embodiment, the at least one substrate concentration for the at least one alcohol oxidase enzyme ranges from 0.01% to 60% by weight, relative to the total weight of the composition. In another exemplary embodiment, the concentration ranges from 0.05% to 30% by weight, relative to the total weight of the composition.

The oxidation bases may be chosen from para-phenylenediamines, bis(phenyl)alkylenedi-amines, para-aminophenols, ortho-aminophenols and heterocyclic bases, and the addition salts thereof.

Among the para-phenylenediamines, mention may be made, by way of example, of para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine and 2-β-hydroxyethylamino-5-aminotoluene, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylene-diamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxy-ethyl)-para-phenylenediamine, 2-chloro-para-phenylenediamine and 2-β-acetylaminoethyloxy-para-phenylenediamine and the addition salts thereof with an acid.

Among the bis(phenyl)alkylenediamines, mention may be made, by way of example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetra-methylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof with an acid.

Among the para-aminophenols, mention may be made, by way of example, of para-aminophenol, 4-amino-3-methylphenol, 4-amino-3-fluorophenol, 4-amino-3-hydroxy-methylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol, 4-amino-2-fluorophenol, 4-amino-2,6-dichlorophenol, 4-amino-6[(5′-amino-2′-hydroxy-3′-methyl)phenylmethyl]-2-methylphenol and bis(5′-amino-2′-hydroxyl)phenylmethane and the addition salts thereof with an acid.

Among the ortho-aminophenols, mention may be made, by way of example, of 2-aminophenol, 2-amino-5-methylphenol, 2-amino-6-methylphenol and 5-acetamido-2-aminophenol, and the addition salts thereof with an acid.

Among the heterocyclic bases, mention may be made, by way of example, of pyridine derivatives, pyrimidine derivatives and pyrazole derivatives.

Among the pyridine derivatives, mention may be made of the compounds described, for example, in patents GB 1 026 978 and GB 1 153 196, such as 2,5-diaminopyridine, 2-(4-methoxyphenyl)amino-3-aminopyridine, and 3,4-diaminopyridine, and the addition salts thereof with an acid. Other pyridine oxidation bases that are useful in the disclosed compositions include the 3-aminopyrazolo[1,5-a]pyridine oxidation bases or the addition salts thereof described, for example, in patent application FR 2 801 308. By way of example, mention may be made of pyrazolo[1,5-a]pyrid-3-ylamine; 2-acetylamino-pyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo[1,5-a]pyrid-7-yl )methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl )(2-hydroxyethyl )amino]ethanol; 2-[(3-aminopyraz-olo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]-pyrid-7-ol; and also the addition salts thereof with an acid.

Among the pyrimidine derivatives, mention may be made of the compounds described, for example, in patents DE 2 359 399; JP 88-169 571; JP 05 163 124; EP 0 770 375 or patent application WO 96/15765, such as 2-hydroxy-4,5,6-triaminopyrimidine, 2,4-dihydroxy-5,6-diaminopyrimidine and pyrazolopyrimidine derivatives such as those mentioned in patent application FR-A-2 750 048, and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid, and the tautomeric forms thereof, when a tautomeric equilibrium exists.

Among the pyrazole derivatives, mention may be made of the compounds described in patents DE 3 843 892, DE 4 133 957 and patent applications WO 94/08969, WO 94/08970, FR-A-2,733,749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethyl pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

In exemplary embodiments, the concentration of the at least one oxidation base ranges from 0.0001% to 20% by weight, relative to the total weight of the composition, such as from 0.005% to 6% by weight, relative to the total weight of the composition.

Oxidation couplers may, for example, include meta-phenylenediamines, meta-aminophenols, meta-diphenols, naphthalenic couplers and heterocyclic couplers, and also the addition salts thereof.

Other examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof.

In exemplary embodiments, the concentration of the at least one oxidation coupler may range from 0.0001% to 20%, by weight, relative to the total weight of the composition, such as from 0.005% to 6% by weight, relative to the total weight of the composition.

The addition salts with an acid that may be used for the oxidation bases and couplers may be chosen from the hydrochlorides, hydrobromides, sulphates, citrates, succinates, tartrates, lactates, tosylates, benzenesulphonates, phosphates and acetates.

The addition salts that may be used in the context of the invention are chosen, for example, from the addition salts with sodium hydroxide, potassium hydroxide, ammonia, amines and alkanolamines.

The composition may also contain at least one direct dye that may be chosen, for example, from neutral, acidic or cationic dyes, neutral, acidic or cationic nitrobenzene dyes, neutral, acidic or cationic azo direct dyes, azine direct dyes and methine, azomethine, triarylmethane and indoamine direct dyes.

The cationic direct dyes that may be used include cationic azo direct dyes described in patent applications WO 95/15144, WO 95/01772 and EP 0 714 954. Among these compounds, exemplary embodiments include the following dyes:
1,3-dimethyl-2-[(4-(dimethylamino)phenyl)azo]-1H-imidazolium chloride;
1,3-dimethyl-2-[(4-(aminophenyl)azo)-1H-imidazolium chloride; and
1-methyl-4-[(methylphenylhydrazono)methyl]pyridinium methyl sulphate.

The at least one direct dye may, for example, have a concentration ranging from 0.001% to 20% by weight, relative to the total weight of the composition, such as from 0.005% to 10% by weight, relative to the total weight of the composition.

The composition may also contain various adjuvants conventionally used in compositions for dyeing keratin fibers, such as antioxidants, penetrating agents, sequestering agents, fragrances, buffers, dispersants, surfactants, conditioners such as, for example, volatile or non-volatile, modified or unmodified silicones, cationic polymers, cations, film-forming agents, thickening polymers, ceramides, preserving agents, opacifiers, vitamins or provitamins.

The above adjuvants may, for example, be present in an amount for each one ranging from 0.01% to 20% by weight, relative to the total weight of the composition.

The person skilled in the art might take care to select this or these optional additional compound(s) such that the advantageous properties intrinsically associated with the composition, as described herein, are not, or are not substantially, adversely affected by the envisaged addition(s).

The medium that is suitable for dyeing keratin fibers, also known as the dye support, may, for example, comprise water or a mixture of water and at least one organic solvent to dissolve the compounds that would not be sufficiently water-soluble. As appropriate, this solvent may be a substrate of the enzyme such as ethanol or isopropanol. It may also be a non-substrate compound of the enzyme such as polyol ethers, for instance 2-butoxyethanol, propylene glycol, propylene glycol monomethyl ether and diethylene glycol monomethyl ether and monoethyl ether or phenoxyethanol, and mixtures thereof.

The solvents may be present in proportions, for example, ranging from 1% to 40% by weight, relative to the total weight of the composition, such as from 5% to 30% by weight, relative to the total weight of the composition.

The pH of the composition may, for example, range from 6 to 11, such as from 7 to 10. It may be adjusted for the desired value using acidifying or basifying agents usually used in the dyeing of keratin fibers, or alternatively using standard buffer systems.

Among the acidifying agents that may be mentioned, for example, are mineral or organic acids, for instance hydrochloric acid, orthophosphoric acid, sulphuric acid, carboxylic acids, for instance acetic acid, tartaric acid, citric acid and lactic acid, and sulphonic acids.

Among the basifying agents, mention may be made to aqueous ammonia, alkyl metal carbonates, alkanolamines such as monoethanolamine, diethanolamine and triethanolamine and also derivatives thereof, sodium hydroxide, potassium hydroxide and the compounds of formula (III) below: 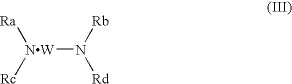
in which W is a propylene residue optionally substituted with a hydroxyl group or a C₁-C₄ alkyl radical; Ra, Rb, Rc and Rd, which may be identical or different, are chosen from a hydrogen atom, C₁-C₄ alkyl and C₁-C₄ hydroxyalkyl radical.

The composition may be in various forms, such as in the form of liquids, creams or gels, or in any other form that is suitable for dyeing keratin fibers, such as human hair.

When the at least one oxidation dye and the at least one alcohol oxidase enzyme are present in the same ready-to-use composition, the composition may be, for example, free of oxygen, so as to avoid any premature oxidation of the at least one oxidation dye.

Another exemplary embodiment disclosed herein includes a process for dyeing keratin fibers, for example human keratin fibers such as the hair, comprising applying at least one composition to the fibers, for a period that is sufficient to develop the desired coloration.

The color is revealed, on contact with atmospheric oxygen, by, for example, bringing the at least one alcohol oxidase enzyme and its at least one substrate into contact.

The composition is applied to the keratin fibers. After leaving it to act, for example for a period of time ranging from 3 to 60 minutes, such as from 5 to 40 minutes, the keratin fibers may be rinsed, washed with shampoo, rinsed again and then dried.

When the composition is in ready-to-use form, it may comprise, in a medium that is suitable for dyeing keratin fibers, at least one oxidation dye precursor, at least one alcohol oxidase enzyme, at least one substrate for the at least one alcohol oxidase enzyme and at least one quinone direct dye; the mixture may be stored in anaerobic form, e.g., free of oxygen gas.

In an exemplary embodiment, a process for dyeing keratin fibers comprising applying to keratin fibers at least one composition comprising, in a medium that is suitable for dyeing keratin fibers: at least one oxidation dye precursor, at least one alcohol oxidase enzyme, at least one substrate having at least one alcohol functional group for the at least one alcohol oxidase enzyme, and at least one quinone direct dye. If the at least one oxidation dye precursor comprises at least one aromatic or aliphatic alcohol functional group, then the substrate may be at least partially replaced by the oxidation dye precursor. In this process, the composition is applied to the keratin fibers for a period that is sufficient to develop the desired coloration.

According to one exemplary embodiment, a process includes a preliminary step that comprises separately storing, on the one hand, a composition (A) comprising in a medium that is suitable for dyeing keratin fibers, at least one oxidation dye precursor, and, a composition (B) comprising, in a medium that is suitable for dyeing keratin fibers, at least one alcohol oxidase enzyme. The composition (A) and/or the composition (B) comprising at least one substrate for the at least one alcohol oxidase enzyme, and the composition (A) and/or the composition (B) comprising at least one quinone direct dye. The process then comprises mixing together the compositions (A) and (B) at the time of use before applying this mixture to the keratin fibers.

According to another exemplary embodiment, a process includes a preliminary step that comprises separately storing, on the one hand, a composition (A) comprising in a medium that is suitable for dyeing keratin fibers, at least one oxidation dye precursor, at least one substrate for the at least one alcohol oxidase enzyme, and at least one quinone direct dye and, a composition (B) comprising, in a medium that is suitable for dyeing keratin fibers, at least one alcohol oxidase enzyme. The process further comprises mixing together the compositions (A) and (B) at the time of use before applying this mixture to the keratin fibers.

The color may be revealed at acidic, neutral or alkaline pH. In the case where the process is performed using a composition (A) comprising at least one oxidation dye precursor, at least one substrate for the at least one alcohol oxidase enzyme and at least one quinone direct dye, and a composition (B) comprising at least one alcohol oxidase enzyme, the enzyme may be added to the composition (A) via the composition (B), just at the time of use, as indicated below, or it may be applied simultaneously or sequentially to the composition (A).

This composition (B), (so-called oxidizing composition) may also comprise various adjuvants conventionally used in compositions for dyeing the hair and as defined herein.

The pH of the composition (B), so-called oxidizing composition, is such that, after mixing with the composition (A), the pH of the resultant composition applied to the keratin fibers ranges, for example, from 6 to 11, such as from 7 to 10. It may be adjusted to the desired value by means of acidifying or basifying agents usually used in the dyeing of keratin fibers and as defined above.

In an exemplary embodiment, the application of the composition according to the invention is carried out at a temperature ranging, for example, from ambient temperature to 220° C., or, for example, ranging from ambient temperature to 60° C.

In another exemplary embodiment is a multi-compartment device or dyeing “kit”, in which a first compartment contains the composition (A) as defined above and a second compartment contains the composition (B) as defined above. This device may be equipped with a means for applying the desired mixture to the hair, such as the devices described in patent FR-2 586 913 in the name of the Applicant.

In exemplary embodiments, processes for dyeing keratin fibers can comprise using at least one of the multi-component devices described herein.

The examples that follow serve to illustrate the invention without, however, being limiting in nature.

### EXAMPLE

The following composition was prepared.

| | | |
|---|---|---|
| Acid Violet 43 (quinone direct dye) | 0.5 | g |
| Ethanol (donor substrate) | 25 | g |
| para-Phenylenediamine (dye precursor) | 3 × 10⁻³ | mol |
| meta-Aminophenol (coupler) | 3 × 10⁻³ | mol |
| Alcohol oxidase | 20000 | units |

| | |
|---|---|
| 2-Amino-2-methyl-1-propanol | qs pH 7 |
| Distilled water | qs 100 g |

The alcohol oxidase used was the product sold by the company Biozyme Laboratories, in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above composition was applied to locks of natural grey permanent-waved hair containing 90% white hairs, and left to act for 30 minutes. The bath ratio was set at 5. The alcohol oxidase was added extemporaneously. The hair was then rinsed, washed with a standard shampoo and then dried.

Hair dyed in a natural shade of dark blonde was obtained.

1. A composition for dyeing keratin fibers comprising, in a medium that is suitable for dyeing:
at least one oxidation dye precursor,
at least one alcohol oxidase enzyme,
at least one substrate having at least one alcohol functional group for the at least one alcohol oxidase enzyme, and
at least one quinone direct dye;
wherein if the at least one oxidation dye precursor comprises at least one aromatic or aliphatic alcohol functional group, then the at least one substrate is optionally partially or fully replaced by the at least one oxidation dye precursor.

2. The composition according to claim 1, wherein the at least one quinone direct dye is chosen from benzoquinone dyes, naphthoquinone dyes, and anthraquinone dyes.

3. The composition according to claim 1, wherein the at least one quinone direct dye is chosen from Disperse Red 15, Solvent Violet 13, Acid Violet 43 (C.I. 60730), Disperse Violet 1, Disperse Violet 4, Disperse Blue 1, Disperse Violet 8, Disperse Blue 3, Disperse Red 11, Acid Blue 25 (C.I. 62055), Acid Blue 62 (C.I. 62045), Acid Blue 78 (C.I. 62105), Disperse Blue 7, Basic Blue 22, Disperse Violet 15, Basic Blue 99, Mordant Red 3 (C.I. 58005), Acid Green 25 (C.I. 61570), N-methylmorpholinium-propylamino-4-hydroxyanthraquinone, 1-aminopropyl-amino-4-methylaminoanthraquinone, 1-aminopropylaminoanthra-quinone, 5-β-hydroxyethyl-1,4-diamino-anthraquinone, 2-aminoethylaminoanthraquinone, 1,4-bis(β,γ-dihydroxy-propylamino)anthraquinone, 2,5-dihydroxybenzoquinone, 2-hydroxy-3-methoxy-naphthoquinone, and 2,3-dihydroxy-naphthoquinone.

4. The composition according claim 1, wherein the at least one quinone direct dye is chosen from natural quinone direct dyes.

5. The composition according to claim 1, wherein the at least one quinone direct dye is chosen from lawsone, juglone, alizarin, purpurin, spinulosin, 2,5-dihydroxy-naphthoquinone, carminic acid, and kermesic acid.

6. The composition according to claim 1, wherein the concentration of the at least one quinone direct dye ranges from 0.001% to 20% by weight, relative to the total weight of the composition.

7. The composition according to claim 1, wherein the at least one alcohol oxidase enzyme belongs to the EC sub-subclass E.C.1.1.3.

8. The composition according to claim 1, wherein the at least one alcohol oxidase enzyme is chosen from primary alcohol oxidases (EC 1.1.3.13), secondary alcohol oxidases (EC 1.1.3.18), long-hydrocarbon-chain alcohol oxidases (EC 1.1.3.20), polyvinyl alcohol oxidases (EC 1.1.3.30), vanillyl alcohol oxidase (EC 1.1.3.38), and aromatic alcohol oxidases (EC 1.1.3.7).

9. The composition according to claim 1, wherein the at least one alcohol oxidase enzyme is obtained from at least one species chosen from Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia sp., Pichia pastoris, Pichia methanolica, Pichia angusta, Candida sp., Candida boidinii, Candida albicans, Candida tropicalis, Pinus strobus, Gastropode mollusc, and Manduca sexta.

10. The composition according to claim 1, wherein the at least one alcohol oxidase enzyme is obtained from Pichia pastoris.

11. The composition according to claim 1, wherein the concentration of the at least one alcohol oxidase enzyme ranges from 0.05% to 20% by weight, relative to the total weight of the composition.

12. The composition according to claim 1, wherein the concentration of the at least one alcohol oxidase enzyme ranges from 10³U to 10⁵U per 100 g of the composition.

13. The composition according claim 1, wherein the at least one substrate comprises an alcohol chosen from branched and unbranched, saturated and unsaturated, and substituted and unsubstituted primary alcohols, secondary alcohols, long-hydrocarbon-chain alcohols, and aromatic alcohols.

14. The composition according to claim 1, wherein the concentration of the at least one substrate ranges from 0.01% to 60% by weight, relative to the total weight of the composition.

15. The composition according to claim 1, wherein the at least one oxidation dye precursor comprises at least one oxidation base chosen from para-phenylenediamines, bis(phenyl)alkylenediamines, para-aminophenols, ortho-aminophenols and heterocyclic bases, and the addition salts thereof.

16. The composition according to claims 15, wherein the concentration of the at least one oxidation base ranges from 0.0001% to 20% by weight, relative to the total weight of the composition.

17. The composition according to claim 1, wherein the at least one oxidation dye precursor comprises at least one oxidation coupler chosen from meta-phenylenediamines, meta-aminophenols, meta-diphenols, naphthalenic couplers and heterocyclic couplers, and the addition salts thereof.

18. The composition according to claim 17, wherein the concentration of the at least one oxidation coupler ranges from 0.0001% to 20% by weight, relative to the total weight of the composition.

19. The composition according to claim 1, wherein the composition further comprises at least one direct dye other than the at least one quinone direct dye.

20. A multi-compartment device comprising
a first compartment comprising a composition (A) comprising, in a medium that is suitable for dyeing keratin fibers, at least one oxidation dye precursor, and
a second compartment comprising a composition (B) comprising, in a medium that is suitable for dyeing keratin fibers, at least one alcohol oxidase enzyme;
wherein at least one of compositions (A) and (B) comprise at least one substrate for the at least one alcohol oxidase enzyme, and at least one composition (A) and (B) comprises at least one quinone direct dye.

21. The multi-compartment device of claim 20, wherein the first compartment comprises at least one oxidation dye precursor, at least one substrate for at least one alcohol oxidase enzyme, and at least one quinone direct dye, and the second compartment comprises at least one alcohol oxidase enzyme.

22. A process for dyeing keratin fibers comprising
applying to keratin fibers at least one dyeing composition for a period sufficient to develop a desired coloration, wherein the dyeing composition comprises, in a medium that is suitable for dyeing,
at least one oxidation dye precursor,
at least one alcohol oxidase enzyme,
at least one substrate having at least one alcohol functional group for the at least one alcohol oxidase enzyme, and
at least one quinone direct dye;
wherein if the at least one oxidation dye precursor comprises at least one aromatic or aliphatic alcohol functional group, then the at least one substrate is optionally partially or fully replaced by the at least one oxidation dye precursor.

23. The process according to claim 22, wherein the at least one composition is at least one ready-to-use composition stored in a form substantially free of oxygen.

24. The process according to claim 22, comprising separately storing a composition (A) comprising in a medium that is suitable for dyeing, at least one oxidation dye precursor, and a composition (B) comprising, in a medium that is suitable for keratin fibres, at least one alcohol oxidase enzyme, wherein the composition (A) and/or the composition (B) comprise at least one substrate for the enzyme and the composition (A) and/or the composition (B) comprise at least one quinone direct dye,
the process further comprising mixing compositions (A) and (B) together at the time of use and before applying to the keratin fibers.
